(19) United States
(12) Patent Application Publication
(10) Pub. No.: US 2007/0202006 A1
(43) Pub. Date: Aug. 30, 2007

(54) Disinfecting Solutions Effective Against Bacterial Endospores

(76) Inventors: Daniel M. Ammon JR., Rochester, NY (US); Roya Nicole Borazjani, Rochester, NY (US); Joseph C. Salamone, Fairport, NY (US)

Correspondence Address: Rita D. Vacca;Baush & Lomb Incorporated, One Baush & Lomb Place, Rochester, NY, 14604-2701, US

(21) Appl. No.: 11/742,743

(22) Filed: May 1, 2007

Related U.S. Application Data
(62) Division of application No. 10/412,795, filed on Apr. 11, 2003, now abandoned.

(51) Int. Cl. A61L 2/16

(52) U.S. Cl. 422/34; 422/243; 422/28; 422/305; 422/37

(57) ABSTRACT

The present invention is directed to a biguanide-containing disinfecting solutions effective in inactivating bacteria endospores on surfaces, air-borne or in water. The methods of using the present invention are directed to disinfecting endospore laden surfaces, air and water with the subject biguanide-containing solutions.

This application is a divisional application of U.S. patent application Ser. No. 10/412,795, which was filed on Apr. 11, 2003.

### BACKGROUND OF THE INVENTION

The present invention is directed to biguanide-containing disinfecting solutions effective as rapid bacterial endosporicidals. More particularly, the present invention is directed to biguanide-containing rapid disinfecting solutions effective against bacterial endospores belonging to the Family of Bacillaceae, such as but not limited to those members of the Genus Bacillus and the Genus Clostridium, the two classified endospore forming bacteria. The present invention is likewise directed to methods for making and using the same.

The most resistant form of life is the bacterial endospore. This stage in the life of certain bacteria is a response to environmental signals that forecast problems for the survival of the vegetative (reproducing) cell. One such signal is a reduction in the water surrounding the cell. The vegetative cell undergoes a series of changes resulting in the formation of metabolically inert endospores, which are highly resistant to heat, chemicals and UV light. The vegetative cell changes include: decrease in internal water concentration; formation of several thick layers or coats that surround the cell's deoxyribonucleic acid (DNA) and a portion of its cytoplasm; and the presence of large amounts of calcium and dipicolinic acid (DPA). The resultant endospore is a dormant form and does not reproduce. Endospores are difficult to kill except by strong chemicals, high heat, or gamma irradiation. When conditions signal a favorable environment, the endospores will germinate and the emergent vegetative cell can resume to replicate.

Although bacteria from about a half dozen genera can form endospores, the most common endospore-formers are members of the Genus Bacillus and the Genus Clostridium. Both are Gram positive rods, but Bacillus are aerobic or facultatively anaerobic whereas Clostridium are anaerobic. The endospore, which may survive for prolonged periods in the environment, may infect and germinate inside the body. For example, the etiological agent of tetanus, Clostridium tetani, will reside in the soil as an endospore, but if it enters the hospitable warmth, moisture and nutrients of a deep (anaerobic) wound, it will return to the actively reproducing vegetative stage.

The Genus Bacillus and the Genus Clostridium each contains organisms that are pathogenic. Such organisms may be infectious through one or more of the following mechanisms of exposure, depending upon the particular type of organism: inhalation, with infection through respiratory mucosa or lung tissues; ingestion; contact with the mucous membranes of the eyes, or nasal tissues; or penetration of the skin through open cuts or abrasions. Chemical disinfection of endospore-forming bacteria and their endospores involves the use of strong chemical agents in the form of a liquid, gas or aerosol.

While many antibacterial and antimicrobial agents are known, few agents are effective in the destruction or inactivation of bacteria endospores. The principal disinfecting agents for destruction or inactivation of bacteria endospores are formaldehyde, glutaraldehyde (at pH 8.0-8.5), hydrogen peroxide and peracetic acid (Dietz and Bohm, 1980; Bohm, 1990). Hypochlorites are sporicidal but are rapidly neutralized by organic matter and, therefore, while good for disinfecting non-wooden surfaces, are unsuitable for disinfecting most environmental sites or materials. Hydrogen peroxide and peracetic acid are not appropriate disinfecting agents if blood is present.

Although effective endospore disinfecting agents, formaldehyde, glutaraldehyde, hydrogen peroxide, peracetic acid and chlorine compounds are toxic to humans and largely dangerous to handle. Formaldehyde (formalin) is poisonous and a suspect cancer hazard. The risk of cancer from formaldehyde depends on the level and duration of exposure. Formaldehyde (formalin) vapor is harmful if inhaled or absorbed through the skin. Formaldehyde is very corrosive and can not be made nonpoisonous. It is also flammable as a liquid and as a vapor. The endospore disinfecting agent glutaraldehyde is likewise very corrosive and harmful if swallowed, inhaled, or absorbed through the skin. Hydrogen peroxide, while less dangerous to handle than formaldehyde and glutaraldehyde, may be harmful if swallowed and is known to cause eye irritation. The endospore disinfectant peracetic acid, like formaldehyde and glutaraldehyde is very corrosive and all contact should be avoided. Peracetic acid is corrosive to the eyes, the skin, the respiratory tract and upon ingestion. Inhalation of peracetic acid may cause lung oedema. It is also flammable and explosive. The endospore disinfectant chlorine dioxide is corrosive by inhalation and can cause serious skin burns. It is also flammable and explosive.

Accordingly, there is a need for improved bacterial endosporicidal agents that rapidly and effectively destroy bacterial endospores and maintain a low order of toxicity in humans.

### SUMMARY OF THE INVENTION

In accordance with this invention, there is provided solutions effective for rapidly disinfecting bacterial endospore laden surfaces, air and/or water comprising bacterial endosporicidally effective amounts of a biguanide or water-soluble salts thereof. The subject biguanide-containing solutions are useful in the rapid destruction of bacterial endospores, are non-toxic to humans for ease in handling, are simple to use and do not cause tissue irritation upon contact. The solutions of the present invention are particularly useful in instances where repeated hand washing and tissue contact is necessary, such as for example with medical care providers, since the subject solutions are non-irritating to tissue.

Accordingly, it is an object of the present invention to provide biguanide-containing solutions useful as bacterial endosporicidal agents.

Another object of the present invention is to provide a method for using biguanide-containing solutions useful as bacterial endosporicidal agents.

Another object of the present invention is to provide a method for making biguanide-containing solutions useful as bacterial endosporicidal agents.

Another object of the present invention to provide biguanide-containing solutions useful as bacterial endosporicidal agents that are non-toxic to humans.

Another object of the present invention to provide biguanide-containing solutions useful as bacterial endosporicidal agents that are easy to handle.

Another object of the present invention is to provide biguanide-containing solutions that do not cause tissue irritation upon contact therewith.

Still another object of the present invention is to provide biguanide-containing solutions with rapid bacterial endosporicidal activity.

These and other objectives and advantages of the present invention, some of which are specifically described and others that are not, will become apparent from the detailed description and claims that follow.

### DETAILED DESCRIPTION OF THE INVENTION

The biguanide-containing solutions of the present invention are useful in killing bacterial endospores on surfaces, in the air or in water. The solutions of the present invention are particularly useful in instances where repeated hand washing and/or tissue contact is necessary, such as with medical care providers, since the same are non-irritating to tissue. “Non-irritating to tissue” means that the subject solutions do not cause adverse skin reactions such as redness, swelling and/or itching or burning sensations upon contact. Given the fact that bacterial endospores are the most resistant form of life, it is surprising that the subject biguanide-containing aqueous solutions are so highly effective as rapid bacterial endosporicidals while being non-toxic to humans. The subject biguanide-containing solutions are also desirable as bacterial endosporicidals in that they have no harmful or offensive fumes.

The biguanide-containing solutions of the present invention comprise at least approximately 100 ppm, but more preferably at least approximately 100 ppm to less than approximately 200,000 ppm of one or more biguanides, and most preferably greater than approximately 1000 ppm to less than approximately 150,000 ppm of one or more biguanides. Suitable “biguanides” as referred to herein include biguanides, bis-biguanides and poly-biguanides. Such biguanides include for example but are not limited to chlorhexidine (1,1′-hexamethylene-bis[5-(p-chlorophenyl)biguanide]) or water soluble salts thereof, such as chlorhexidine gluconate, poly(hexamethylene biguanide) or water soluble salts thereof, such as poly(hexamethylene biguanide) hydrochloride, and 1,1′-hexamethylene-bis[5-(2-ethylhexyl)biguanide] (Alexidine) and chlorhexidine. Biguanides are described in U.S. Pat. No. 5,990,174 incorporated herein in its entirety by reference. The preferred biguanide due to its ready commercial availability and superior endosporicidal effectiveness is poly(aminopropyl biguanide) (PAPB), also commonly referred to as poly(hexamethylene biguanide) (PHMB). The subject solutions preferably likewise comprise deionized water and one or more buffers.

The pH of the subject solutions can range between 4.0 to 9.0 but will generally range between about 6.0 to about 8.0, and more preferably between 6.5 to 7.5. As mentioned above, one or more buffers may be employed to obtain the desired pH value. Tromethamine (tris(hydroxymethyl)aminomethane, (TRIS)) is a known buffer, which is suitable for use in the present invention. As an alternative, the subject solutions may include a supplemental buffering agent, meaning that the subject solutions may include a “mixed buffer” of tromethamine and one or more other buffer agents. Other suitable buffers include for example but are not limited to borate buffers such as but not limited to boric acid, potassium tetraborate, potassium metaborate, sodium borate and mixtures thereof, phosphate buffers such as but not limited to Na₂HPO₄, NaH₂PO₄, KH₂PO₄ and mixtures thereof, citrate buffers such as for example but not limited to sodium citrate, potassium citrate, citric acid and mixtures thereof, sodium bicarbonate, amino alcohol buffers and combinations thereof. Preferably, solutions of the present invention include TRIS and sodium borate in combination with sodium chloride or glycerin. One or more buffers are preferably added to solutions of the present invention in amounts ranging between approximately 0.01 to 2.0 weight percent by volume, but more preferably between approximately 0.05 to 0.5 weight percent by volume.

Solutions of the present invention may be designed for a variety of osmolalities. Preferably, solutions in accordance with the present invention have an osmotic value of less than about 350 mOsm/kg, more preferably from about 175 to about 330 mOsm/kg, and most preferably from about 175 to about 320 mOsm/Kg. One or more osmolality adjusting agents may be employed in the subject solutions to obtain the desired final osmolality. Suitable osmolality adjusting agents include for example but are not limited to sodium and potassium chloride, monosaccharides such as dextrose, calcium and magnesium chloride, and low molecular weight polyols such as glycerin and propylene glycol. Typically, these agents are used individually in amounts ranging from about 0.01 to 5 weight percent and preferably, from about 0.1 to about 2 weight percent.

Preferably, the subject solutions likewise include one or more poly(ethylene oxide) (PEO) containing materials, which serve to assist in solubilization and hydration. Suitable PEO-containing materials include for example but are not limited to certain polyethyleneoxy-polypropyleneoxy block copolymers, also known as poloxamers. Such materials are commercially available under the trade name Pluronic™ and Tetronic™ (BASF Corporation, Parsippany, N.J.), e.g., Tetronic 904, Tetronic 1307, Pluronic F127, Pluronic 105 and Pluronic 123. One or more PEO-containing materials are preferably added to solutions of the present invention in amounts ranging between approximately 1.0 to 20 weight percent by volume, but more preferably between approximately 10 to 15 weight percent by volume and most preferably approximately 12 weight percent by volume. Suitable PEO-containing materials likewise include for example N-alkyl PEO monoethers and a material commercially available under the trade name Brij™ 700 (ICI Americas Corporation, Edison, N.J.).

Preferably, the subject solutions likewise include one or more wetting agents, which serve to increase binding to surfaces by hydrogen-bonding interactions, hydrophobic interactions, and where appropriate, ionic interactions. Suitable wetting agents include for example but are not limited to cellulosic materials including cationic cellulosic polymers, hydroxypropyl methylcellulose, hydroxyethyl cellulose and methylcellulose, poly(vinyl alcohol), and poly(N-vinylpyrrolidone). A preferred class of wetting agent is the cationic cellulosic materials that have the ability to associate with anionic areas on surfaces. Such materials include polyquaternium-10 available under the trade name Polymer JR™-30 M (ICI Americas Corporation). Other wetting agents include quaternized guar gums such as guar hydroxypropyltrimonium chloride and hydroxypropyl guar hydroxypropyltrimonium chloride, and particularly products available under the trade name Jaguar™ (Rhodia, Cranberry, N.J.). Such wetting agents may be used in a wide range of concentrations in the solutions of the present invention, generally within the range of approximately 0.01 to 10 weight percent by volume.

Optionally, the subject solutions may likewise include one or more additional surfactants to effect surface cleaning. Suitable surfactants include for example but are not limited to cationic, anionic, nonionic and amphoteric surfactants. Preferred surfactants are nonionic and amphoteric surfactants. The surfactants used in the present invention should be soluble in the aqueous solution and non-irritating to tissues.

Suitable non-ionic surfactants include for example polyethylene glycol esters of fatty acids, e.g., coconut, polysorbate, polyoxyethylene or polyoxypropylene ethers of higher alkanes (C₁₂-C₁₈), polysorbate 20 available under the trademark Tween® 20 (ICI Americas Corporation), polyoxyethylene (23) lauryl ether available under the trademark Brij® 35, (ICI Americas Corporation), polyoxyethyene (40) stearate available under the trademark Myrj® 52, (ICI Americas Corporation) and polyoxyethylene (25) propylene glycol stearate available under the trademark Atlas® G 2612 (ICI Americas Corporation).

Amphoteric surfactants suitable for use in solutions according to the present invention include materials of the type offered commercially under the trademark Miranol™ (Rhodia). Another useful class of amphoteric surfactants is exemplified by cocoamidopropyl betaine, commercially available from various sources.

Various other ionic as well as amphoteric and anionic surfactants suitable for use in the present invention are described in McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

Preferably, one or more surfactants, when present in the subject solutions, are employed in a total amount from about 0.01 to about 17 weight percent, preferably 0.1 to 15 weight percent, and most preferably 0.1 to 12 weight percent.

One or more chelating agents can also be added to the subject solutions. Suitable chelating agents include for example but are not limited to hydroxyalkyl-phosphonates as disclosed in U.S. Pat. No. 5,858,937 (Richards et al.), and available under the tradename Dequest® (Monsanto Co., St. Louis, Mo.).

Test formulations of biguanide-containing solutions in accordance with the present invention, and in some instances non-biguanide-containing solutions, are set forth in Table 1 below.

TABLE 1
Bacterial Endosporicidal Test Solutions

| Components | Concentration | Soln. 1 | Soln. 2 | Soln. 3 | Soln. 4 | Soln. 5 | Soln. 6 | Soln. 7 | Soln. 8 |
|---|---|---|---|---|---|---|---|---|---|
| Water | QS to 100% | X | X | X | X | X | X | X | X |
| Tris Base | 0.12% | X | X | X | X | X | X | X | X |
| Na Borate | 0.22% | X | X | X | X | X | X | X | X |
| Polymer JR | 0.04% | X | X | X | X | X | X | X | X |
| Alexidine | 10 ppm | X | X | X | X | X | X | X | X |
| PHMB | x ppm | 5 | 5 | 5 | 1000 | 100 | 20 | 10 | 1 |
| Tetronic 904 | 3% | X | X | X | X | X | X | X | X |
| Tetronic 1307 | 3% | X | X | X | X | X | X | X | X |
| Pluronic F127 | 1% | X | X | X | X | X | X | X | X |
| Pluronic 105 | 1% | X | X | X | X | X | X | X | X |
| Pluronic123 | 1% | X | X | X | X | X | X | X | X |
| BRIJ | 3% | X | X | X | X | X | X | X | X |
| Osmolality (mOsm/kg) | | 206 | 200 | 195 | 199 | 199 | 199 | 199 | 199 |
| pH | | 7.00 | 7.00 | 7.01 | 7.03 | 7.03 | 7.03 | 7.03 | 7.03 |
| Components | Concentration | Soln. 9 | Soln. 10 | Soln. 11 | Soln. 12 | Soln. 13 | Soln. 14 | Soln. 15 | Soln. 16 |
| Water | QS to 100% | X | X | X | X | X | X | X | X |
| Tris Base | 0.12% | X | X | X | X | X | X | X | X |
| Na Borate | 0.22% | X | X | X | X | X | X | X | X |
| Polymer JR | x% | 0.04 | 0.08 | 0.12 | 0.24 | 0.50 | 0.04 | 0.04 | — |
| Alexidine | 10 ppm | X | X | X | X | X | X | X | — |
| PHMB | x ppm | 5 | 5 | 5 | 5 | 5 | 100 | 100 | — |
| Tetronic 904 | 3% | X | X | X | X | X | X | — | X |
| Tetronic 1307 | 3% | X | X | X | X | X | X | — | X |
| Pluronic F127 | 1% | X | X | X | X | X | X | — | X |
| Pluronic 105 | 1% | X | X | X | X | X | X | — | X |
| Pluronic 123 | 1% | X | X | X | X | X | X | — | X |
| BRIJ | 3% | X | X | X | X | X | X | — | X |
| Glycerin | 1.2% | — | — | — | — | — | — | X | — |
| Osmolality (mOsm/kg) | | 212 | 212 | 212 | 212 | 212 | 201 | 199 | 208 |
| pH | | 6.95 | 6.95 | 6.95 | 6.95 | 6.95 | 6.97 | 6.97 | 7.09 |

| Components | Concentration | Soln. 17 | Soln. 18 | Soln. 19 | Soln. 20 | Soln. 21 | Soln. 22 |
|---|---|---|---|---|---|---|---|
| Water | QS to 100% | X | X | X | X | X | X |
| Tris Base | 0.12% | X | X | X | X | X | X |
| Na Borate | 0.22% | X | X | X | X | X | X |
| Polymer JR | 0.04% | X | X | X | X | X | X |
| Alexidine | 10 ppm | X | X | X | X | X | X |
| PHMB | 100 ppm | X | X | X | X | X | X |
| Tetronic 904 | 12% | X | — | — | — | — | — |
| Tetronic1307 | 12% | — | X | — | — | — | — |
| Pluronic F127 | 12% | — | — | X | — | — | — |
| Pluronic 105 | 12% | — | — | — | X | — | — |
| Pluronic 123 | 12% | — | — | — | — | X | — |
| BRIJ | 12% | — | — | — | — | — | X |
| Osmolality (mOsm/kg) | | 234 | 231 | 195 | 206 | 213 | 215 |
| pH | | 7.00 | 7.00 | 7.00 | 7.00 | 7.00 | 7.00 |

| Components | Concentration | Soln. 23 | Soln. 24 | Soln. 25 | Soln. 26 | Soln. 27 | Soln. 28 | Soln. 29 |
|---|---|---|---|---|---|---|---|---|
| Water | QS to 100% | X | X | X | X | X | X | X |
| Tris Base | 0.12% | X | X | X | X | X | X | X |
| Na Borate | 0.22% | X | X | X | X | X | X | X |
| Polymer JR | 0.04% | X | — | — | X | X | — | X |
| Alexidine | 10 ppm | — | X | — | X | — | X | X |
| PHMB | 100 ppm | — | — | X | — | X | X | X |
| Tetronic 904 | 3% | X | X | X | X | X | X | X |
| Tetronic 1307 | 3% | X | X | X | X | X | X | X |
| Pluronic F127 | 1% | X | X | X | X | X | X | X |
| Pluronic 105 | 1% | X | X | X | X | X | X | X |
| Pluronic 123 | 1% | X | X | X | X | X | X | X |
| BRIJ | 3% | X | X | X | X | X | X | X |
| Osmolality (mOsm/kg) | | 198 | 190 | 195 | 192 | 194 | 203 | 195 |
| pH | | 7.00 | 7.00 | 7.00 | 6.9 | 7.00 | 7.00 | 7.00 |

| Components | Concentration | Soln. 30 | Soln. 31 | Soln. 32 | Soln. 33 | Soln. 34 | Soln. 35 | Soln. 36 | Soln. 37 |
|---|---|---|---|---|---|---|---|---|---|
| Water | QS to 100% | X | X | X | X | X | X | X | X |
| Tris Base | x% | 0.25 | 0.25 | 0.14 | 0.14 | — | — | — | — |
| Na Borate | x% | — | — | 0.22 | 0.22 | 0.018 | 0.018 | — | — |
| NaCl | x% | 0.51 | — | 0.50 | — | 0.50 | — | 0.45 | — |
| Glycerine | x% | — | 1.52 | — | 1.32 | — | 1.60 | — | 1.25 |
| PHMB | 100 ppm | X | X | X | X | X | X | X | X |
| Boric Acid | 0.12% | — | — | — | — | X | X | — | — |
| Na Phos. (m) | 0.08% | — | — | — | — | — | — | X | X |
| Na Phos. (di) | 0.39% | — | — | — | — | — | — | X | X |
| Osmolality (mOsm/kg) | | 196 | 197 | 223 | 208 | 190 | 197 | 218 | 227 |
| pH | | 6.9 | 6.98 | 7.08 | 6.98 | 6.99 | 7.02 | 7.00 | 7.04 |

| Components | Concentration | Soln. 38 | Soln. 39 | Soln. 40 | Soln. 41 | Soln. 42 | Soln. 43 |
|---|---|---|---|---|---|---|---|
| Water | QS to 100% | X | X | X | X | X | X |
| Tris Base | 0.12% | X | X | X | X | X | X |
| Na Borate | 0.22% | X | X | X | X | X | X |
| Polymer JR | 0.04% | — | — | — | — | — | X |
| Alexidine | x ppm | 5 | 10 | 20 | 50 | 100 | 100 |
| PHMB | 100 ppm | — | — | — | — | — | X |
| Tetronic 904 | 3% | X | X | X | X | X | X |
| Tetronic 1307 | 3% | X | X | X | X | X | X |
| Pluronic F127 | 1% | X | X | X | X | X | X |
| Pluronic 105 | 1% | X | X | X | X | X | X |
| Pluronic 123 | 1% | X | X | X | X | X | X |
| BRIJ | 3% | X | X | X | X | X | X |
| Osmolality (mOsm/kg) | | 185 | 183 | 180 | 185 | 180 | 191 |
| pH | | 6.92 | 6.96 | 7.17 | 7.03 | 6.95 | 7.00 |

| Components | Concentration | Soln. 44 | Soln. 45 | Soln. 46 | Soln. 47 | Soln. 48 | Soln. 49 | Soln 50 |
|---|---|---|---|---|---|---|---|---|
| Water | QS to 100% | X | X | X | X | X | X | X |
| Tris Base | 0.12% | X | X | X | X | X | X | X |
| Na Borate | 0.22% | X | X | X | X | X | X | X |
| Polymer JR | 0.04% | X | X | X | X | X | X | X |
| Alexidine | 10 ppm | X | X | X | X | X | X | X |
| PHMB | x ppm | 100 | 100 | 100 | 100 | 100 | 100 | 1000 |
| Tetronic 904 | 3% | X | X | X | X | X | X | X |
| Tetronic 1307 | 3% | X | X | X | X | X | X | X |
| Pluronic F127 | 1% | X | X | X | X | X | X | X |
| Pluronic 105 | 1% | X | X | X | X | X | X | X |
| Pluronic 123 | 1% | X | X | X | X | X | X | X |
| BRIJ | 3% | X | X | X | X | X | X | X |
| Osmolality (mOsm/kg) | | 195 | 201 | 197 | 194 | 188 | 185 | 199 |
| pH | | 4.00 | 5.00 | 6.00 | 7.00 | 8.00 | 9.00 | 7.03 |

X = the amount provided under “concentration” or that amount specified for the particular test solution
— = component not added to solution

The biguanide-containing bacterial endosporicidal solutions of the present invention are described in still greater detail in the examples that follow.

### EXAMPLE 1

### Determination of Minimal Inhibitory Concentration of Selected Test Solutions Against 10³ Endospores in Modified Trypticase Soy Broth on Cellulose Membrane

The object of the present study was to determine the minimal inhibitory concentration of selected test solutions against 10³ endospores in Modified Trypticase Soy Broth (MTSB) on cellulose membrane. To do this, 0.1 ml of a 10⁴ suspension of Bacillus stearothermophilus endospores in 50 mM phosphate buffer with a pH of 7.0, was added to 20 ml of sterile phosphate buffer and the entire contents were then filtered through a 0.2 μm filter. Filters containing endospores (10³ spores/membrane) were then placed on absorbent pads, which had been previously soaked with 1.8 ml of solution containing diluted test solution (2, 10, 50 and 100-fold dilutions) in MTSB. The pads/filters were then placed in individual petri dishes and incubated at 55° C. Recovery of spores was examined after both 24 hours and 48 hours.

Materials used in the subject study are listed below.
1. Test solutions 3-7, 14-29 and a 1:1 mixture of solutions 23 and 24
2. 50 mM phosphate buffer, pH 7.0
   Sodium phosphate monobasic—NaH₂PO₄ (Sigma # S-0751, Sigma Chemical Company, St. Louis, Mo.)
   Sodium phosphate dibasic—Na₂HPO₄ (Fisher # S-374, Fisher Scientific, Swanee, Ga.)
3. Modified TSB medium for spore recovery and growth of vegetative cells
   Bacto™ Tryptic Soy Broth (TSB), (DIFCO #211825, Lot #0341002, Becton, Dickinson, & Co., Sparks, Md.)
   Sodium thiosulfate—Na₂S₂O₃-5H₂O (Fisher #S-445)
   Tween™ 80 (J. T. Baker, #X257-07, Mallinckrodt, St. Louis, Mo. and Baker, Inc., Phillipsburg, N.J.)
4. Certified spore suspension of B. stearothermophilus, 2×10⁷ spores/ml (STS, Sterilization Technical Services, Rush, N.Y.)
5. 15 ml polypropylene conical tube (Falcon, #352097, Becton, Dickinson, & Co. Sparks, Md.)
6. Sterile Petri Dishes
7. 47 mm sterile cellulose pads
8. Sterile 0.2μ filters (Millipore, Billerica, Mass.)
9. 6-place filter manifold systems (Gelman, Pall Corporation, East Hills, N.Y.)
10. 47 mm vacuum filter units (Pall, Pall Corporation, East Hills, N.Y.)
11. A vacuum pump
12. Refrigerated incubator shaker (Innova 4230, New Brunswick Scientific, Edison, N.J.)

In the subject study, dilutions of the test solutions (2-, 10-, 50- and 100-fold dilutions) were made from the original test solutions into MTSB. Test solution 4 was diluted 10-fold prior to making the above dilutions in MTSB. Duplicate 1.8 ml aliquots of the diluted test solutions in MTSB were added to 47 mm sterile cellulose pads contained in sterile petri dishes. Individual 0.1 ml aliquots of diluted B. stearothermophilus spore solution (1.0×10⁴ spores/ml) were added to 20 ml buffer and the entire contents were filtered through a 0.2 micron membrane filter. Individual membrane filters were then transferred to petri dishes containing the prepared sterile cellulose pads. Membranes were slightly bent when placed on the wetted pads to ensure proper contact, and then rolled onto the pad to eliminate bubbles and voids. The petri dishes were incubated at 55° C. Recovery was examined after 24 and 48 hours of incubation. A control with 1.8 ml MTSB only on the cellulose pad was also examined. The results of the subject study are set forth below in Table 2.

TABLE 2
Ranking of activity based upon recovery of B. stearothermophilus
on cellulose membranes after incubation for 48 hours at 55° C.

| | Dilutions | | | |
|---|---|---|---|---|
| Solution | 2 | 10 | 50 | 100 |

| | | | | | | | | |
|---|---|---|---|---|---|---|---|---|
| 14 | − | − | − | − | − | − | + | + |
| 27 | − | − | − | − | − | − | + | + |
| 15 | − | − | − | − | 1 | 1 | + | + |
| 25 | − | − | − | − | 1 | 1 | + | + |
| 20 | − | − | − | − | 1 | 1 | + | + |
| 18 | − | − | − | − | 2 | 1 | + | + |
| 4 | − | − | − | − | 4 | 2 | + | + |
| 22 | − | − | − | − | 3 | 11 | + | + |
| 19 | − | − | − | − | 6 | 3 | + | + |
| 5 | − | − | − | − | 5 | 5 | + | + |
| 21 | − | − | − | − | 10 | 10 | + | + |
| 17 | − | − | − | − | 8 | 15 | + | + |
| 28 | − | − | − | − | 22 | 5 | + | + |
| 29 | − | − | − | − | 52 | 58 | + | + |
| 6 | − | − | 47 | 49 | + | + | + | + |
| 7 | − | − | 136 | + | + | + | + | + |
| 3 | − | − | + | + | + | + | + | + |
| 24 | − | − | + | + | + | + | + | + |
| 23 + 24 | − | − | + | + | + | + | + | + |
| 26 | − | − | + | + | + | + | + | + |
| 16 | + | + | + | + | nd | + | + | + |
| 23 | + | + | + | + | + | + | + | + |
| control | | | | + | + | | | |

+ = >150 cfu/membrane
− = no growth
nd = not determined

According to the above study, none of the test solutions was effective against 10³ spores when diluted 100-fold with MTSB. A summary of the ranking of test solutions based upon recovery after 48 hours at 55° C. is set forth
Group 1: Effective up to 50-fold dilution—Solutions 14 and 27.
Group 2: Moderately effective at 50-fold dilution, having slight break-through in growth with <10 cfu recovered—Solutions 15, 25, 20, 18, 4, 22, 19 and 5.
Group 3: Partially effective at 50-fold dilution, having break-through in growth with >10 but <60 cfu recovered—
   Solutions 21, 17, 28 and 29.
Group 4: Partially effective at 10-fold dilution, having break-through in growth with >50 cfu recovered—
   Solutions 6 and 7.
Group 5: Effective at a 2-fold dilution—
   Solutions 3, 24, 23+24 and 26.
Group 6: Ineffective at a 2-fold dilution (no biguanide present)—
   Solutions 23 and 16.

### EXAMPLE 2

### Determination of Anti-Microbial Activity for Selected Solutions Against 10⁴ Spores of B. globgii (Membrane Recovery Test)

Solutions to be evaluated were diluted with Modified Trypticase Soy Broth (MTSB) and then 10⁴ B. globigii spores were added to the diluted test solutions. Recovery of spores (as measured by outgrowth of vegetative cells) was determined after incubation at 30° C. for 24 hours. Solutions 16 and 14 were used as controls. The ranking of solutions so tested, set forth below in Tables 4A and 4B, is based upon recovery after 24 hours at 30° C. When considering the results summarized in Tables 4A and 4B below, it should be noted that B. globigii grows much faster than B. stearothermophilus. Also, the criteria used for the ranking of solutions in Tables 4A and 4B as set forth below in Table 3 are the same criteria used to rank activity against B. stearothermophilus spores. Overall, rankings tend to be better for Minimal Inhibitory Concentration (MIC) testing the membrane testing.

TABLE 3
Criteria Used for Ranking Endosporicidal Test Solutions

| | |
|---|---|
| Group 1: | Effective at >100 fold dilution. 1++ (33 and 32)/1+ (31)/1 |
| | (Solutions 45, 49, 44, 14 and 47) |
| Group 2: | Moderately effective at 50-fold dilution (Solutions 48 and 46) |
| Group 3: | Partially effective at 50-fold dilution (Solutions 35, 36, 37, 34, |
| | 42, and 30) |
| Group 4: | Partially effective at 10-fold dilution. |
| Group 5: | Effective at a 2-fold dilution |
| Group 6: | Ineffective at any dilution tested (Solutions 41, 38, 40, 39 |
| | and 16) |

TABLE 4A
RANKING OF ANTI-MICROBIAL ACTIVITY
AGAINST ENDOSPORES OF
B. globigii: MEMBRANE RECOVERY TEST
(Recovery after 24 hours)

Dilutions

| Solution | 2 | 10 | 50 | 100 | Group rank |
|---|---|---|---|---|---|
| 31 | −/− | −/− | 1/− | 1/− | 1 |
| 33 | −/− | −/− | −/−* | +/+ | 1 |
| 32 | −/− | −/− | −/−* | +/+ | 1 |
| 30 | −/− | −/− | −/−* | +/+ | 1 |
| 14 | −/− | −/− | −/−* | +/+ | 1 |
| (− control) | | | | | |
| 36 | −/− | −/− | −/−* | +/+ | 1 |
| 35 | −/− | 1/5 | −/− | +/+ | 1 |
| 46 | −/− | 11/14* | −/− | +/+ | 1 |
| 44 | −/− | 22/42 | +/+ | +/+ | 2 |
| 47 | −/− | 15/18 | +/+ | +/+ | 2 |
| 49 | −/− | 10/25 | +/+ | +/+ | 3 |
| 41 | −/− | −/1 | +/+ | +/+ | 3 |
| 40 | −/− | 1/1 | +/+ | +/+ | 3 |
| 42 | −/− | 1/1 | +/+ | +/+ | 3 |
| 48 | −/− | 19/25 | +/+ | +/+ | 3 |
| 45 | −/− | 41/71 | +/+ | +/+ | 3 |
| 37 | −/− | 13/+ | +/+ | +/+ | 6 |
| 33 | −/− | 30/+ | +/+ | +/+ | 6 |
| 38 | −/− | +/+ | +/+ | +/+ | 6 |
| 39 | −/− | +/+ | +/+ | +/+ | 6 |
| 16 | +/+ | +/+ | +/+ | +/+ | 6 |
| (+control) | | | | | |

*No growth at 24 hours, but growth noted at 48 hours.

TABLE 4B
RANKING OF ANTI-MICROBIAL ACTIVITY
AGAINST ENDOSPORES OF
B. globigii: Based upon 24 h recovery MIC

| | Dilutions | | | | | |
|---|---|---|---|---|---|---|
| Solution | 50 | 100 | 200 | 400 | 800 | Group rank |
| 33 | −/− | −/− | −/− | −/− | +/+ | 1++ |
| 32 | −/− | −/− | −/− | −/− | +/+ | 1++ |
| 31 | −/− | −/− | −/− | +/+ | +/+ | 1+ |
| 45 | −/− | −/− | +/+ | +/+ | +/+ | 1 |
| 49 | −/− | −/− | +/+ | +/+ | +/+ | 1 |
| 41 | −/− | −/− | +/+ | +/+ | +/+ | 1 |
| 14 | −/− | −/− | +/+ | +/+ | +/+ | 1 |
| 47 | −/− | −/− | +/+ | +/+ | +/+ | 1 |
| 48 | −/− | −/+ | +/+ | +/+ | +/+ | 2 |
| 46 | −/− | +/− | −/− | +/+ | +/+ | 2 |
| 35 | −/− | +/+ | +/+ | +/+ | +/+ | 3 |
| 36 | −/− | +/+ | +/+ | +/+ | +/+ | 3 |
| 37 | −/− | +/+ | +/+ | +/+ | +/+ | 3 |
| 34 | −/− | +/+ | +/+ | +/+ | +/+ | 3 |
| 42 | −/− | +/+ | +/+ | +/+ | +/+ | 3 |
| 30 | −/− | +/+ | +/+ | +/+ | +/+ | 3 |
| 41 | +/+ | +/+ | +/+ | +/+ | +/+ | 6 |
| 38 | +/+ | +/+ | +/+ | +/+ | +/+ | 6 |
| 40 | +/+ | +/+ | +/+ | +/+ | +/+ | 6 |
| 39 | +/+ | +/+ | +/+ | +/+ | +/+ | 6 |
| 16 | +/+ | +/+ | +/+ | +/+ | +/+ | 6 |
| +control | +/+ | +/+ | +/+ | +/+ | +/+ | |
| −control | −/− | −/− | −/− | −/− | −/− | |

No growth at 24 hours, but growth noted at 48 hours.

### EXAMPLE 3

### Efficacy of Selected Test Solutions Against a Dried Film Containing Bacillus globigii Endospore (5 Minute Exposure)

The objective of this study was to determine the efficacy of selected test solutions against 5×10⁶ spores of Bacillus globigii on a glass surface. In this study, the ASTM (American Society for Testing and Methods) standard method for efficacy of sanitizers recommended for inanimate non-food contact surfaces, E-1153-94, was used but modified to assess the activity against endospores of B. globigii rather than the dried cells of Klebsiella as specified by the test. To do this, to the surface of previously cleaned and sterilized glass coupons (25×25 mm), 20 μl of a 2.5×10⁸ spores/ml spore suspension of Bacillus globigii endospores was added and then spread over the surface of the coupon within 3 mm of the edge. The film was then allowed to air dry. Note: The ASTM test calls for drying at 35% relative humidity (RH). As the level of performance in this new test could not be anticipated, drying was accomplished under ambient conditions in a sterile hood. The test coupons were then exposed to 5.0 ml of a selected test solution at room temperature for 5 minutes. The same was then neutralized by the addition of 20 ml of sterile Dye-Engly neutralizing broth (D/E) followed by standardized mixing, i.e., jars containing the coupons were rotated in a single plane only, for 30 seconds at 300 rpm. The number of viable endospores remaining after treatment was enumerated by standard plate count on TSA plate. Numbers of surviving, viable endospores were determined by the enumeration of colonies (cfu) of vegetative cells after 24 hours incubation at 30° C.

Materials used in the subject study are listed below.
1. Test solutions 4, 14, 41, 42, 43 and 61.
2. 50 mM phosphate buffer, pH 7.0
   Sodium phosphate monobasic—NaH₂PO₄ (Sigma # S-0751, Sigma Chemical Company, St. Louis, Mo.)
   Sodium phosphate dibasic—Na₂HPO₄ (Fisher # S-374, Fisher Swanee, Ga.)
3. Difco™ Tryptic Soy Agar (TSA), (DIFCO #236950, Lot #2190630, Becton, Dickinson, & Co., Sparks, Md.)
4. Certified spore suspension of B. globigii, 2.5×10⁸ spores/ml (STS, Sterilization Technical Services, Rush, N.Y.)
5. Sterile petri dishes, 60×15 mm, polystyrene (Falcon, #3351007, Becton, Dickinson, & Co., Sparks, Md.)
6. Glass slide cut to 25×25×1 mm, polished and washed (VWR #48300-025)
7. Wide-mouth polypropylene autoclavable jars, 60 ml (Fisher #02891B, Swanee, Ga.)
8. Refrigerated incubator (Innova™ 4230, New Brunswick Scientific, Edison, N.J.)
9. Mixer/titer plate shaker (Lab-Line Instruments, Inc., Melrose Park, Ill.)
10. Laminar hood, Class II, type A, biohazard cabinet (Envirco Corporation, Albuquerque, N. Mex.)
11. Dye-Engly Broth (D/E) Difco (Becton, Dickinson, & Co., Sparks, Md.)

In the subject study, all manipulations were conducted within a sterile hood unless otherwise specified. To conduct the study, a single, cleaned, sterile, glass square (25×25 mm) was placed into a sterile 60 mm petri dish. To each coupon, 20 μl of a 2.5×10⁸ spores/ml B. globigii spore suspension was added and then spread to within 3 mm of the edge of the coupon. The inoculated coupons were then air-dried overnight. The individual coupons were then transferred to sterile 60 ml jars. Test solutions were then prepared by i) making a 10-fold serial dilution of sample by transferring a 0.1 ml aliquot of neutralized test solution to 0.9 ml phosphate buffer, ii) transferring duplicate 0.1 ml aliquots from two appropriate dilution levels to TSA plates and spreading, and iii) transferring triplicate 0.01 ml aliquots from 4 dilution levels to TSA plates. Control A solutions were then prepared by i) making a 10-fold serial dilution of sterile water by transferring 0.1 ml aliquot of neutralized sterile water to 0.9 ml phosphate buffer, ii) transferring duplicate 0.1 ml aliquots from two appropriate dilution levels to TSA plates and spreading, and iii) transferring triplicate 0.01 ml aliquots from 4 dilution levels to TSA plates. Control B solutions were then prepared by i) transferring 0.02 ml of a 2.5×10⁸ spores/ml B. globigii spore suspension to a 25 ml solution containing 5 ml sterile water and 20 ml D/E neutralizing broth, ii) making a 10-fold serial dilution by transferring 0.1 ml aliquot to 0.9 ml phosphate buffer, iii) transferring duplicate 0.1 ml aliquots from two appropriate dilution levels to TSA plates and spreading, and iv) transferring triplicate 0.01 ml aliquots from 4 dilution levels to TSA plates. 5 ml of test solution, or the control, was added to each coupon so as to cover the inoculated coupon. The coupons were then allowed to stand for 5 minutes. This procedure was carried out in triplicate for each test solution. 20 ml of D/E neutralizing broth was added to each jar to neutralize the test solution. The jars were then rotated, in a single plane only, for 30 seconds at 300 revolutions per minute (rpm). The plates were incubated at 30° C. for 24 hours, at which time the colonies were examined and enumerated. The study results are set forth in Table 5 below.

TABLE 5
Efficacy of Selected Test Solutions Against a Dried Film Containing
Bacillus globigii Endospore After 5 Minutes

| | Survived spores | Log | | Std. | Log reduction |
|---|---|---|---|---|---|
| Solution | per coupon (ss/c) | (ss/c) | Avg. | Dev. | in 5 minutes |
| 4 | 0.00E+00 | 0.00 | 0.00 | 0.00 | 6.09 |
| | 0.00E+00 | 0.00 | | | |
| | 0.00E+00 | 0.00 | | | |
| 14 | 1.88E+03 | 3.27 | 3.95 | 0.60 | 2.14 |
| | 1.38E+04 | 4.14 | | | |
| | 2.74E+04 | 4.44 | | | |
| 31 | 1.25E+05 | 5.10 | 5.15 | 0.26 | 0.94 |
| | 8.33E+04 | 4.92 | | | |
| | 2.67E+05 | 5.43 | | | |
| 32 | 4.60E+04 | 4.66 | 4.86 | 0.20 | 1.23 |
| | 1.17E+05 | 5.07 | | | |
| | 7.24E+04 | 4.86 | | | |
| 33 | 1.92E+05 | 5.28 | 5.44 | 0.16 | 0.65 |
| | 4.08E+05 | 5.61 | | | |
| | 2.67E+05 | 5.43 | | | |
| 43 | 8.00E+03 | 3.90 | 4.69 | 0.70 | 1.40 |
| | 8.78E+04 | 4.94 | | | |
| | 1.73E+05 | 5.24 | | | |
| Control A* | 5.05E+06 | 6.70 | 6.71 | 0.01 | NA |
| | 5.29E+06 | 6.72 | | | |
| Control B** | 1.67E+06 | 6.22 | 6.09 | 0.18 | NA |
| | 9.17E+05 | 5.96 | | | |

*Control A spore suspension inoculated with 0.02 ml of 2.5e8 spores/ml was added to 0.9 ml phosphate buffer and was regarded as the first 10-fold dilution (dilution level −1).
**Control B non-treated glass inoculated with 0.02 ml of 2.5e8 spores/ml then treated as test samples except that 5.0 ml water, instead of test solution was used.
NA = Not Applicable.

### EXAMPLE 4

### Efficacy of Selected Test Solutions and Disinfectants Against a Dried Film Containing Bacillus globigii Endospore (1 Minute Exposure)

An identical study to that of Example 3 was conducted with the exception that exposure of the endospores to the test solutions, disinfectants and controls was for 1 minute rather than for 5 minutes. The study results are set forth in Tables 6A and 6B below.

TABLE 6A
Efficacy of Selected Test Solutions Against a Dried Film Containing
Bacillus globigii Endospore After 1 Minute

| | Survived spores | Log | | Std. | Log reduction |
|---|---|---|---|---|---|
| Solution | per coupon (ss/c) | (ss/c) | Avg. | Dev. | in 1 minute |
| 4 | 7.00E+04 | 4.85 | 4.42 | 1.17 | 2.15 |
| | 1.25E+03 | 3.10 | | | |
| | 2.10E+05 | 5.32 | | | |
| 14 | 2.84E+05 | 5.45 | 5.61 | 0.21 | 0.96 |
| | 7.00E+05 | 5.85 | | | |
| | 3.48E+05 | 5.54 | | | |
| 31 | 1.75E+05 | 5.24 | 5.44 | 0.17 | 1.13 |
| | 3.50E+05 | 5.54 | | | |
| | 3.42E+05 | 5.53 | | | |
| 32 | 3.09E+05 | 5.49 | 5.56 | 0.06 | 1.01 |
| | 3.88E+05 | 5.59 | | | |
| | 3.96E+04 | 5.60 | | | |
| 33 | 8.33E+05 | 5.92 | 5.57 | 0.31 | 1.00 |
| | 3.00E+05 | 5.48 | | | |
| | 2.08E+05 | 5.32 | | | |
| 43 | 2.28E+05 | 5.36 | 5.05 | 0.35 | 1.53 |
| | 4.63E+04 | 4.67 | | | |
| | 1.28E+05 | 5.11 | | | |
| Control A* | 6.42E+06 | 6.81 | 6.73 | 0.11 | NA |
| | 4.42E+06 | 6.65 | | | |
| Control B** | 3.67E+06 | 6.56 | 6.57 | 0.01 | NA |
| | 3.75E+06 | 6.57 | | | |

*Control A spore suspension inoculated with 0.02 ml of 2.5e8 spores/ml was added to 0.9 ml phosphate buffer and was regarded as the first 10-fold dilution (dilution level −1).
**Control B non-treated glass inoculated with 0.02 ml of 2.5e8 spores/ml then treated as test samples except that 5.0 ml water, instead of test solution was used.
NA = Not Applicable.

TABLE 6B
Efficacy of Disinfectants Against a Dried Film Containing
Bacillus globigii Endospore After 1 Minute

| | Survived | Log (Survived | | | |
|---|---|---|---|---|---|
| | spores | spores | | Std. | Log |
| Disinfectant | per coupon | per coupon) | Average | Dev. | Reduction |
| 1000 ppm | 8.38E+04 | 4.92 | 4.75 | 0.21 | 1.70 |
| | 6.63E+04 | 4.82 | | | |
| | 3.25E+04 | 4.51 | | | |
| 100 ppm | 8.38E+04 | 4.92 | 5.00 | 0.40 | 1.45 |
| | 2.71E+05 | 5.43 | | | |
| | 4.38E+04 | 4.64 | | | |
| Ctrl-A | 2.86E+06 | 6.46 | 6.52 | 0.09 | N/A |
| | 3.85E+06 | 6.59 | | | |
| Ctrl-B | 1.64E+06 | 6.21 | 6.45 | 0.33 | N/A |
| | 4.85E+06 | 6.69 | | | |

1) Sporicidal efficacy of diluted samples of disinfectant at 1.0 minute exposure time was less than that achieved when Solution 4 was tested using a 1.0 minute exposure. This result supports the contention that the efficacy of the “active ingredient” is improved when the active ingredient is properly formulated.
2) It was noted during the recovery of endospores after the challenge that the neutralizing solution (D/E) was not fully effective in neutralizing the diluted disinfectant.

### EXAMPLE 5

### Efficacy of Selected Test Solutions Against a Dried Film Containing Bacillus globigii Endospore (30 Seconds Exposure)

An identical study to that of Example 3 was conducted with the exception that exposure of the endospores to the test solutions and controls was for 30 seconds rather than for 5 minutes. The study results are set forth in Table 7 below.

TABLE 7
Efficacy of Selected Test Solutions Against a Dried Film Containing
Bacillus globigii Endospore After 30 Seconds

| | Survived spores | Log | | Std. | Log reduction |
|---|---|---|---|---|---|
| Solution | per coupon (ss/c) | (ss/c) | Avg. | Dev. | in 30 Seconds |
| 4 | 3.43E+05 | 5.53 | 5.35 | 0.28 | 1.09 |
| | 1.06E+05 | 5.03 | | | |
| | 3.13E+05 | 5.49 | | | |
| 14 | 4.93E+05 | 5.69 | 5.80 | 0.15 | 0.64 |
| | 9.39E+05 | 5.97 | | | |
| | 5.38E+05 | 5.73 | | | |
| 31 | 6.69E+05 | 5.83 | 5.91 | 0.19 | 0.53 |
| | 1.35E+05 | 6.13 | | | |
| | 6.03E+05 | 5.78 | | | |
| 32 | 6.70E+05 | 5.83 | 5.78 | 0.39 | 0.66 |
| | 2.34E+05 | 5.37 | | | |
| | 1.42E+06 | 6.15 | | | |
| 33 | 8.18E+05 | 5.91 | 5.54 | 0.32 | 0.90 |
| | 2.50E+05 | 5.40 | | | |
| | 2.09E+05 | 5.32 | | | |
| 43 | 3.04E+05 | 5.48 | 5.54 | 0.09 | 0.90 |
| | 3.19E+05 | 5.50 | | | |
| | 4.44E+05 | 5.65 | | | |
| Control A* | 3.24E+06 | 6.50 | 6.60 | 0.13 | NA |
| | 4.85E+06 | 6.69 | | | |
| Control B** | 2.93E+06 | 6.47 | 6.45 | 0.04 | NA |
| | 2.61E+06 | 6.42 | | | |

*Control A spore suspension inoculated with 0.02 ml of 2.5e8 spores/ml was added to 0.9 ml phosphate buffer and was regarded as the first 10-fold dilution (dilution level −1).
**Control B non-treated glass inoculated with 0.02 ml of 2.5e8 spores/ml then treated as test samples except that 5.0 ml water, instead of test solution was used.
NA = Not Applicable.

### EXAMPLE 6

### Efficacy of Selected Test Solution and Disinfectant Against a Dried Film Containing Bacillus globigii Endospore (30 Seconds and 1 Minute Exposure)

An identical study to that of Example 3 was conducted with the exception that exposure of the endospores to the test solution, disinfectant (25 ppm=0.0025% w/v) and controls was for 30 seconds and 1 minute rather than for 5 minutes. The study results are set forth in Table 8 below.

TABLE 8
Efficacy of Selected Test Solutions Against a Dried Film Containing
Bacillus globigii Endospore After 30 Seconds and 1 Minute

| | | | | | Log reduction | |
|---|---|---|---|---|---|---|
| | Survived spores | Log | | Std. | 30 | |
| Solution | per coupon (ss/c) | (ss/c) | Avg. | Dev. | sec | 1 min |
| PHMB 20 w % | 0 | 0 | 0 | n/a | 6.47 | 6.47 |
| | 0 | 0 | 0 | n/a | 6.47 | 6.47 |
| | 0 | 0 | 0 | n/a | 6.47 | 6.47 |
| 50 | 0 | 0 | 0 | n/a | 6.47 | 6.47 |
| | 0 | 0 | 0 | n/a | 6.47 | 6.47 |
| | 0 | 0 | 0 | n/a | 6.47 | 6.47 |
| Control A* | 2.88E+06 | 6.46 | 6.54 | 0.11 | n/a | |
| | 4.10E+06 | 6.61 | | | | |
| Control B** | 4.00E+06 | 6.60 | 6.47 | 0.18 | n/a | |
| | 2.21E+06 | 6.34 | | | | |

*Control A spore suspension inoculated with 0.02 ml of 2.5e8 spores/ml was added to 0.9 ml phosphate buffer and was regarded as the first 10-fold dilution (dilution level −1).
**Control B non-treated glass inoculated with 0.02 ml of 2.5e8 spores/ml then treated as test samples except that 5.0 ml water, instead of test solution was used.
NA = Not Applicable.

The present invention also contemplates that the bacterial endosporicidal solutions of the present invention may be employed as pharmaceutical agents to be advantageously employed in combating and/or treating infections. Thus, such pharmaceutical agents may be employed to reduce infection, kill microbes, inhibit microbial growth or otherwise abrogate the deleterious effects of microbial infection.

Particular examples of pharmaceutically acceptable forms of the subject solutions include for example but are not limited to oral, eye, topical or nasal spray or in any other form effective to deliver active components of the present invention to a site of microorganism infection. The route of administration is preferably designed to obtain direct contact of the active components of the present invention with the infecting microorganisms.

For topical applications, an acceptable carrier in any of a number of different forms may be used such as for example but not limited to a liquid, cream, foam, lotion or gel. Topical formulations of the subject solutions may additionally comprise organic solvents, emulsifiers, gelling agents, moisturizers, stabilizers, time release agents, dyes, perfumes, and like components commonly employed in formulations for topical administration.

In yet another embodiment, the subject solutions can be used in the personal health care industry in deodorants, soaps, acne/dermatophyte treatment agents and treatments for halitosis.

In other embodiments of the present invention, the subject solutions may be formulated as ready-to-use solutions or may be concentrated or formulated for later mixing with water or an appropriate carrier prior to use, as may be desired for a particular application. Such solutions and/or concentrates may advantageously be packaged and placed in kits with additional useful adjunct items for use against microbial infections, for decontamination and the like. In this case, the solution or concentrate container means may itself be an inhalant, eye dropper or other apparatus for application to decontaminate an infected area. Also included in such kits may be other adjunct items or instruments useful for assisting in the use of the subject solutions for decontamination, microbial infections or the like. Kits of the present invention typically also include a means for containing the contents of the kit in close confinement for commercial sale.

While the invention has been described herein in conjunction with specific examples thereof, this is illustrative only. Accordingly, many alternatives, modifications, and variations will be apparent to those skilled in the art in the light of the foregoing description and it is, therefore, intended to embrace all such alternatives, modifications, and variations as to fall within the spirit and scope of the appended claims.

We claim:

1. A method of killing bacterial endospores comprising contacting a surface contaminated with said endospores with a composition comprising 0.01 wt. % to 15 wt. % of one or more biguanides.

2. The method of claim 2 wherein the bacterial endospores are selected from the group consisting of Bacillus and Clostridium.

3. The method of claim 1 wherein the one or more biguanides are selected from the group consisting of alexidine, chlorhexidine, poly(hexamethylene biguanide) and the corresponding salts thereof.

4. The method of claim 1 wherein the composition further comprises one or more surfactants.

5. The method of claim 4 wherein the one or more surfactants are selected from the group consisting of cationic and nonionic surfactants.

6. The method of claim 4 wherein the one or more surfactants are selected from the group consisting of poloxomers, reverse poloxomers, poloxamines, reverse poloxamines and alkylethylene oxide-containing surfactants.

7. The method of claim 1 wherein the composition further comprises one or more buffers to maintain the pH of the composition from 4.0 to 9.0.

8. The method of claim 1 wherein the one or more biguanides is present from 0.1 wt. % to 15 wt. %.

9. The method of claim 3 wherein the one or more biguanides is present from 0.1 wt. % to 15 wt. %.

10. The method of claim 1 wherein the composition further comprises one or more buffers.

11. The method of claim 1 wherein the composition in contact with the surface contaminated with bacterial endospores exhibits at least a one log reduction after one minute exposure against a dried film containing the endospores.

12. A method of disinfecting hands against bacterial endospores comprising contacting the hands with a composition comprising 0.01 wt. % to 15 wt. % of one or more biguanides.

13. The method of claim 12 wherein the bacterial endospores are selected from the group consisting of Bacillus and Clostridium.

14. The method of claim 12 wherein the one or more biguanides are selected from the group consisting of alexidine, chlorhexidine, poly(hexamethylene biguanide) and the corresponding salts thereof.

15. The method of claim 12 wherein the composition further comprises one or more buffers.

16. A kit for killing bacterial endospores comprising:
a container for holding a composition comprising 0.01 wt. % to 15 wt. % of one or more biguanides, and
an instrument for applying the composition to an infected environment.

17. The kit of claim 16 wherein the instrument is used to release an aerosol to the infected environment.
